(19) United States
(12) Patent Application Publication
(10) Pub. No.: US 2024/0100305 A1
(43) Pub. Date: Mar. 28, 2024

(54) GUIDE WIRE

(71) Applicants: FUJIFILM Healthcare Corporation, Kashiwa-shi, Chiba (JP); FMD Co., Ltd., Tokyo (JP)

(72) Inventors: Hirozumi Takeshima, Tokyo (JP); Tomohiko Tanaka, Tokyo (JP); Hiroshi Kuribara, Chiba (JP); Hironori Kawasaki, Tokyo (JP)

(21) Appl. No.: 18/368,393

(22) Filed: Sep. 14, 2023

(30) Foreign Application Priority Data
Sep. 28, 2022 (JP) 2022-155072

(51) Int. Cl. A61M 25/09

(52) U.S. Cl. CPC A61M 25/09; A61M 2025/09083

(57) ABSTRACT

A guide wire is provided with a core shaft, a coil portion, a sound source, and wiring. The coil portion is coupled to a distal end portion of the core shaft and is helically wound around a periphery of the core shaft to the rearward. The coil portion includes an open portion in which an interval of element wires constituting the coil portion itself is larger than that in another portion. The sound source is provided in an inner side of the open portion. The wiring extends along an extending direction of the core shaft, and a distal end of the wiring is positioned at an inner side of the coil portion. The wiring supplies energy to the sound source.

### CROSS REFERENCE TO RELATED APPLICATION

This application claims priority to Japanese Patent Application No. 2022-155072 filed on Sep. 28, 2022, which is incorporated herein by reference in its entirety including the specification, claims, drawings, and abstract.

### TECHNICAL FIELD

The present disclosure relates to a guide wire, and specifically relates to a guide wire including a helical coil portion at a distal end portion thereof.

### BACKGROUND

Surgeries have been performed by inserting a guide wire into a patient's blood vessel, and inserting a tubular catheter over the guide wire into the blood vessel while the catheter is being navigated by the guide wire inside the catheter. After the catheter has been inserted into the blood vessel, the catheter is used to perform an input of a drug and a procedure using a wire-shaped medical device.

Generally, a structural device for making the procedure easy is applied to the guide wire. For example, in guide wires described in PATENT DOCUMENTS 1 to 3 below, a coil portion having a helical structure is provided to a distal end portion of the guide wire. With the coil portion, the distal end portion of the guide wire is easily advanced along the blood vessel. In the guide wire described in PATENT DOCUMENT 1, a sound source is attached to a distal end of the coil portion. The ultrasound generated from the sound source is detected by an ultrasonic diagnostic apparatus, whereby a position of the distal end portion of the guide wire is obtained. The guide wire in the blood vessel is generally observed by an X-RAY imaging apparatus; however, in a case where a contrast agent cannot be input into the blood vessel and other cases, as described in PATENT DOCUMENT 1, the guide wire in the blood vessel is observed using the ultrasound in some cases. In the guide wire described in PATENT DOCUMENT 3, a sensor is attached near the coil portion. The guide wire is inserted into the blood vessel, whereby the sensor is disposed in the blood vessel.

### CITATION LIST

PATENT DOCUMENT 1: JP 2020-185363 A
PATENT DOCUMENT 2: JP 2012-279 A
PATENT DOCUMENT 3: JP 2015-96231 A

### SUMMARY

As described in PATENT DOCUMENT 1, when the sound source is attached to the distal end of the coil portion, in some cases the mechanical strength of the guide wire is lowered, as compared with a normal guide wire that is provided with no sound source. This is because the sound source is a device for generating ultrasound and does not necessarily include a structure to make the mechanical strength at the distal end of the guide wire equivalent to that of the normal guide wire. Moreover, it is considered that when the sound source is disposed in the inner side of the coil portion, the propagation of the ultrasound that is generated by the sound source is prevented by element wires of the coil portion, so that the ultrasound is not sufficiently emitted to the periphery of the guide wire, and a position of the guide wire is difficult to obtain using the ultrasound that is generated by the sound source.

An object of the disclosure is to enhance the mechanical strength of a guide wire that is provided with a sound source, and to emit the ultrasound to the periphery of the guide wire more reliably.

A guide wire according to the disclosure includes: a core shaft; a coil portion that is coupled to a distal end portion of the core shaft and is helically wound around a periphery of the core shaft; and a sound source that is provided in an inner side of the coil portion, in which the coil portion includes an open portion in which an interval of element wires constituting the coil portion itself is larger than that in another portion.

In one embodiment, the sound source is provided in an inner side of the open portion.

In one embodiment, the coil portion has a bent shape that is bent relative to a longitudinal direction of the guide wire, and the open portion is formed in a range toward a rear from the bent portion in the coil portion.

In one embodiment, the guide wire includes wiring that extends along an extending direction of the core shaft, in which a distal end of the wiring is positioned at the inner side of the coil portion, and the wiring supplies energy to the sound source.

In one embodiment, the coil portion has a bent shape that is bent relative to the longitudinal direction of the guide wire, and the distal end of the wiring is present rearward of the bent portion in the coil portion.

In one embodiment, the wiring is an optical fiber, and the guide wire further includes a light absorber that is provided in the inner side of the coil portion, in which the sound source is formed of the light absorber.

In one embodiment, the wiring is a conductive wire, the sound source is a vibration element, and the vibration element is connected to a distal end of the conductive wire.

Moreover, a guide wire according to the disclosure includes: a core shaft; a coil portion that is coupled to a distal end portion of the core shaft, and is helically wound around a periphery of the core shaft; an optical fiber that extends along an extending direction of the core shaft, and includes a distal end portion that is positioned at an inner side of the coil portion; and a light absorber that covers a surface of the coil portion, in which the coil portion includes an open portion in which an interval of element wires constituting the coil portion itself is larger than that in another portion, and a distal end of the optical fiber is positioned at an inner side of the open portion.

In one embodiment, the coil portion has a bent shape that is bent relative to a longitudinal direction of the guide wire, and the distal end of the optical fiber is present rearward of the bent portion in the coil portion.

In one embodiment, the coil portion has a bent shape that is bent relative to the longitudinal direction of the guide wire, and the open portion is formed in a range toward a rear from the bent portion in the coil portion.

In one embodiment, a gap between the element wires in the open portion is 0.13 mm or more and 0.33 mm or less.

In one embodiment, the guide wire includes a resin filled in the inner side of the coil portion.

With the disclosure, it is possible to enhance the mechanical strength of a guide wire that is provided with a sound source, and to emit the ultrasound to the periphery of the guide wire more reliably.

### BRIEF DESCRIPTION OF DRAWINGS

Embodiment(s) of the present disclosure will be described based on the following figures, wherein:

FIG. 1 is a diagram schematically illustrating a guide wire;

FIG. 2 is a diagram schematically illustrating an axial cross section of the guide wire;

FIG. 3 is a graph illustrating an experiment result obtained by measuring an ultrasonic sound pressure to be output laterally from the guide wire;

FIG. 4 is a diagram schematically illustrating an axial cross section of a guide wire according to a first modification example; and

FIG. 5 is a diagram schematically illustrating an axial cross section of a guide wire according to a second modification example.

### DESCRIPTION OF EMBODIMENTS

An embodiment of the present disclosure will be described with reference to the respective drawings. The same components illustrated in the plurality of the drawings are assigned the same reference numerals, and repeated descriptions thereof are omitted.

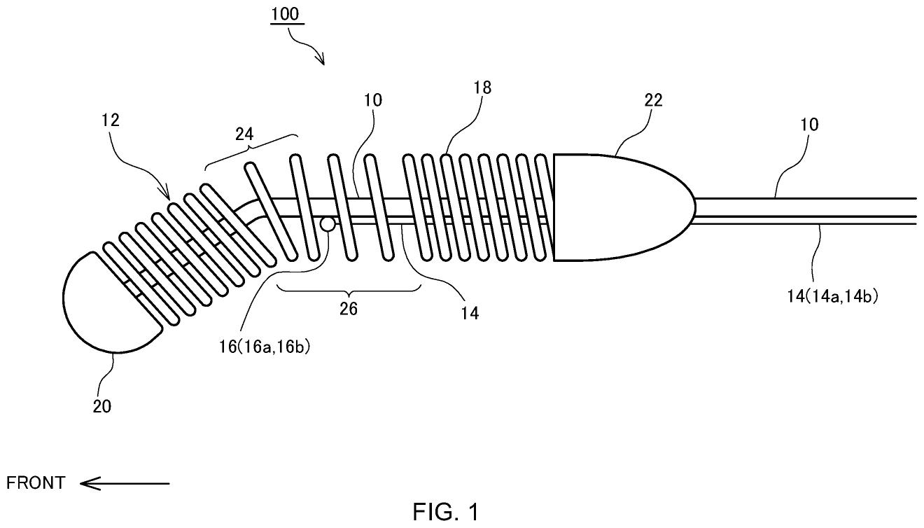

FIG. 1 schematically illustrates a guide wire 100 according to an embodiment of the disclosure. The guide wire 100 is provided with a core shaft 10, a coil portion 12, wiring 14, and a sound source 16. The core shaft 10 is formed of a straightened wire including a publicly known material. The coil portion 12 is formed in such a manner that element wires 18 made of publicly known metal are helically wound. Although no particular limitations are imposed on the materials for the core shaft 10 and the element wires 18, a part of the coil portion 12 is formed of a metal material having X-RAY impermeability to thereby enable a position check of the guide wire by an X-RAY imaging apparatus, similar to a conventional guide wire. A distal end of the coil portion 12 is coupled to a distal end portion of the core shaft 10, and the element wires 18 of the coil portion 12 are helically wound around the periphery of the core shaft 10 to the rearward. The distal end portion of the core shaft 10 and the distal end of the coil portion 12 are coupled to each other with a distal end coupling portion 20. The distal end coupling portion 20 may be a coupling material having rigidity, for example, metal such as solder, stainless steel, iron, aluminum, or copper.

A rear end of the coil portion 12 is fixed to the core shaft 10 with a rear end coupling portion 22. The rear end coupling portion 22 includes, for example, a fixing material such as a plastic resin or solder. Note that, the rear end coupling portion 22 does not need to be provided. In other words, the rear end of the coil portion 12 may be a free end that can swing freely in a front-and-rear direction or a radial direction.

A bent portion 24 is formed at a position nearer to the distal end of the coil portion 12. The bent portion 24 has a shape that is bent in an approximate “L” character shape at a predetermined angle relative to a longitudinal direction of the guide wire 100. The bent portion 24 is provided, for example, in order to cause an operator to easily select one of two blood vessels bifurcated in a blood vessel bifurcated portion, toward which the guide wire 100 is advanced. In the coil portion 12, an open portion 26 in which an interval of the element wires 18 constituting the coil portion 12 itself is larger than that in another portion is formed. In the open portion 26, a pitch in a helical structure that is formed by the element wires 18 is larger than that in another portion. In the example illustrated in FIG. 1, the open portion 26 is formed in a range toward a rear from the bent portion 24. The open portion 26 may include the bent portion 24, or may be disposed in front of the bent portion 24.

The wiring 14 is arranged side by side to the core shaft 10, and extends along an extending direction of the core shaft 10 from the rear toward the front. The wiring 14 penetrates through the rear end coupling portion 22, and a distal end of the wiring 14 is positioned at the inner side of the coil portion 12. The distal end of the wiring 14 is disposed to an inner side of the open portion 26 rearward of the bent portion 24. The sound source 16 is provided to the distal end of the wiring 14. The sound source 16 is provided at a position in the inner side of the open portion 26, in the region in the inner side of the coil portion 12.

The sound source 16 generates ultrasound by energy supplied by the wiring 14. A frequency range of the ultrasound that is generated by the sound source 16 may be a range that can be detected by a medical ultrasonic diagnostic apparatus and the like. The frequency range of the ultrasound that is generated by the sound source 16 may be, for example, 1 MHz or higher and 30 MHz or lower, but is not limited to this range.

The sound source 16 in the present embodiment is a light absorber 16a that expands due to a rise in temperature when absorbing light, and the wiring 14 is an optical fiber 14a. The optical fiber 14a supplies a pulse laser to the light absorber 16a serving as the sound source 16. The light absorber 16a generates ultrasound utilizing the photoacoustic effect. Specifically, the light absorber 16a absorbs the energy of the pulse laser light, and generates ultrasound due to adiabatic expansion by the energy.

The sound source 16 may be a vibration element 16b in which a piezoelectric material is used. In this case, a pair of conductive wires 14b is used as the wiring 14. The vibration element 16b is connected to a distal end of the pair of the conductive wires 14b, and the pair of the conductive wires 14b apply a voltage including a pulse-shaped time waveform to the vibration element 16b serving as the sound source 16. The vibration element 16b generates the ultrasound depending on the applied voltage.

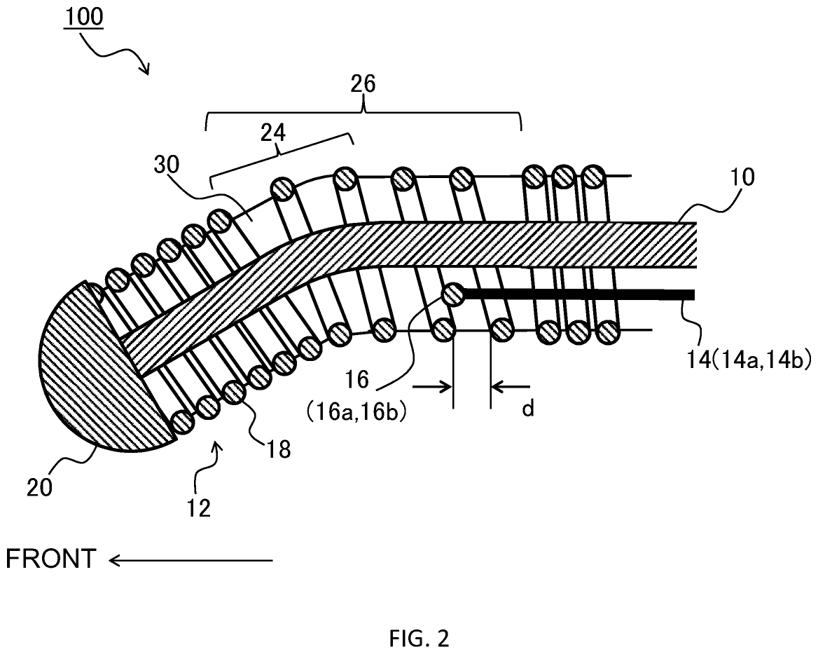

FIG. 2 schematically illustrates a cross section (axial cross section) that appears when the guide wire 100 is cut by a plane parallel to the longitudinal direction. The inner side of the coil portion 12 is filled with a resin 30 such as plastic. With the filling of the resin 30, the core shaft 10, the wiring 14, and the sound source 16 are fixed to the inside of the coil portion 12. Although FIG. 2 illustrates an example in which the inner side of the coil portion 12 is filled with the resin 30, the inner side of the coil portion 12 may be further filled with the resin 30 in a state where the coil portion 12 is molded from the outer side. The open portion 26 is filled with the resin 30 to maintain a gap d between the element wires 18 constant.

An outer diameter of the coil portion 12 may be, for example, 0.3 mm or more and 1.0 mm or less, but is not limited to this range. The gap d between the element wires 18 in the open portion 26 may be, for example, 0.13 mm or more and 0.33 mm or less, but is not limited to this range. The diameter of the coil portion 12 and the gap d between the element wires 18 in the open portion 26 may be determined in accordance with the easiness of the operation of the guide wire 100, characteristics of the ultrasound that is emitted from the guide wire 100, and like factors. Note that, the gap d is set to 0.33 mm or less to suppress a mechanical load to be applied to the open portion 26 during the manipulation, and to prevent kink, deformation, and the like.

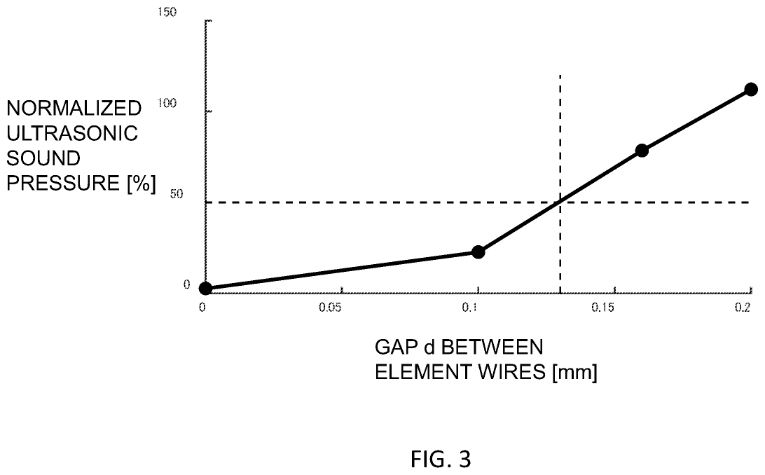

FIG. 3 illustrates an experimental result obtained by measuring an ultrasonic sound pressure to be output laterally from the guide wire 100. The horizontal axis indicates the gap d between the element wires 18, and the longitudinal axis indicates a normalized ultrasonic sound pressure (normalization ultrasonic sound pressure). The normalization ultrasonic sound pressure indicates the percentage of the ultrasonic sound pressure emitted from the guide wire 100 with respect to the ultrasonic sound pressure emitted from a single sound source 16 that is not disposed in the inside of the coil portion 12. In the experiment result illustrated in FIG. 3, when the gap d between the element wires 18 in the open portion 26 is 0.13 mm or more, the normalization ultrasonic sound pressure is 50% or higher. Here, although there is a condition in which the normalization ultrasonic sound pressure exceeds 100%, this is because the sound source 16 is disposed in the inside of the coil portion 12 so that the ultrasound to be emitted in an axis direction of the wiring 14 is reflected on the coil structure, and is emitted laterally.

An example of a procedure using the guide wire 100 will be described. A tubular introducer is inserted from a skin surface toward a blood vessel in a patient, and the guide wire 100 is inserted into the blood vessel through the introducer. With the guide wire 100 inserted into the blood vessel, a tubular catheter is inserted over the guide wire 100 into the blood vessel while the catheter is being navigated by the guide wire 100 inside the catheter. The guide wire 100 in the blood vessel may be observed by an X-RAY imaging apparatus. However, in a case where a contrast agent cannot be input into the blood vessel and in other cases, as indicated in PATENT DOCUMENT 1, the operator may check a position of the sound source 16 on a display of the ultrasonic diagnostic apparatus.

In the guide wire 100 according to the present embodiment, the coil portion 12 is coupled to the core shaft 10 with the distal end coupling portion 20. The distal end coupling portion 20 is formed of a coupling material having rigidity, such as metal. Therefore, the coil portion 12 is reliably fixed to the core shaft 10, the mechanical strength at the distal end of the coil portion 12 is equivalent to that of a normal guide wire that is provided with no conventional sound source, and is thus improved, as compared with a guide wire that is provided with the conventional sound source. Moreover, the coil portion 12 has a bent shape, and includes a region at the distal end side from the bent portion 24 that extends in a different direction with respect to a region at the rear end side thereof. Therefore, the operator easily selects one of two blood vessels bifurcated in a blood vessel bifurcated portion, toward which the guide wire 100 is advanced.

In addition, the open portion 26 is provided in the coil portion 12, and the sound source 16 is disposed to the inner side of the open portion 26. Therefore, in the ultrasound emitted from the sound source 16, the ratio of the ultrasound whose propagation is prevented by the element wires 18 of the coil portion 12 is reduced, so that the ultrasonic sound pressure to be emitted from the guide wire 100 is increased. Moreover, in the open portion 26, the gap between the element wires 18 becomes wider than that in another portion, over the periphery of the coil portion 12. Therefore, the ultrasound is emitted over a wide angular range seen from the coil portion 12. Accordingly, the ultrasound emitted from the sound source 16 is reliably detected by the ultrasonic diagnostic apparatus, so that the operator easily grasps a position of the guide wire 100.

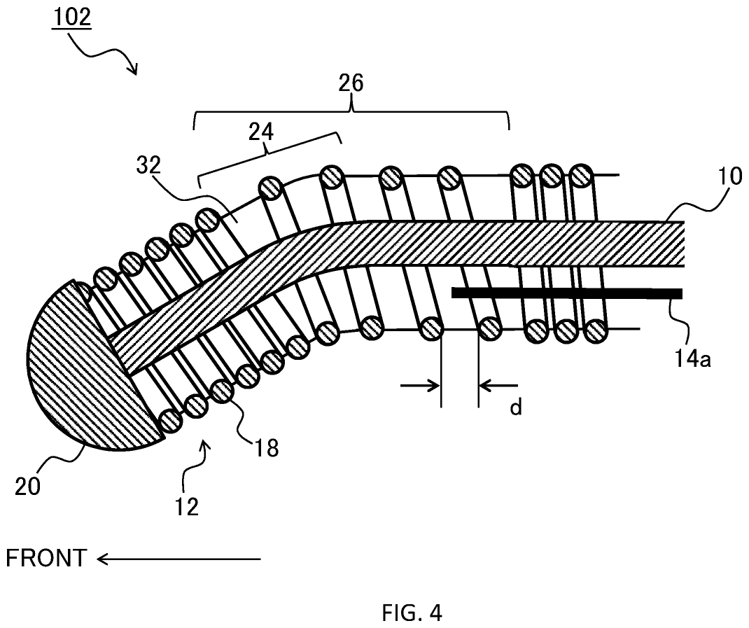

FIG. 4 illustrates an axial cross-sectional diagram of a guide wire 102 according to a first modification example in the disclosure. In the guide wire 102, the optical fiber 14a is provided as the wiring 14 in the guide wire 100 illustrated in FIG. 2, and the inner side of the coil portion 12 is filled with a light absorber 32. In the guide wire 102, the sound source 16 having a form of a device is not used. The optical fiber 14a transfers a pulse laser, and the pulse laser is released from a distal end of the optical fiber 14a. In a region of the light absorber 32, a region near the distal end of the optical fiber 14a operates as a sound source. In other words, the light absorber 32 present near the distal end of the optical fiber 14a adiabatically expands in response to the pulse laser, and generates ultrasound.

In the guide wire 102, in the region of the light absorber 32 filled in the inner side of the coil portion 12, the region near the distal end of the optical fiber 14a is used as a sound source. With the guide wire 102, an effect similar to an effect that is obtained by the guide wire 100 in FIGS. 1 and 2 can be obtained.

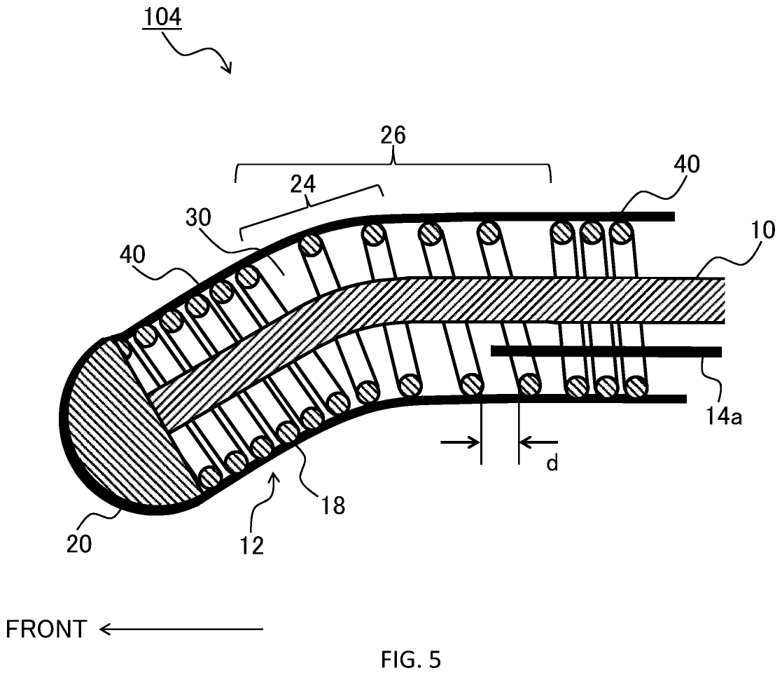

FIG. 5 illustrates an axial cross-sectional diagram of a guide wire 104 according to a second modification example in the disclosure. In the guide wire 104, the optical fiber 14a is provided as the wiring 14 in the guide wire 100 illustrated in FIGS. 1 and 2, the sound source 16 in a form of a device is removed, and a surface of the coil portion 12 is covered with a light absorber 40. The light absorber 40 may be introduced from the gap between the element wires 18 constituting the coil portion 12 to the inner side of the coil portion 12.

The optical fiber 14a transfers a pulse laser, and the pulse laser is released from the distal end of the optical fiber 14a. From the distal end of the optical fiber 14a, the pulse laser is emitted from the inside of the coil portion 12 to the outer side. The light absorber 40 is irradiated with the pulse laser in the gaps between the element wires 18 of the coil portion 12, and the light absorber 40 emits ultrasound.

In the guide wire 104 according to the present modification example, the open portion 26 is provided to the coil portion 12, and the distal end of the optical fiber 14a is positioned at the inner side of the open portion 26. With such a structure, in the pulse laser emitted from the optical fiber 14a, the ratio of the pulse laser whose propagation is prevented by the element wires 18 of the coil portion 12 is reduced, as compared with a case where no open portion 26 is provided. Therefore, the ultrasonic sound pressure emitted by the guide wire 104 is increased, as compared with a case where no open portion 26 is provided, so that an effect similar to that of the guide wire 100 can be obtained.

In the foregoing, the guide wires 100, 102, and 104 according to the embodiment of the disclosure have been indicated. A guide wire according to the disclosure may include the following configurations.

(Configuration 1)

A guide wire includes: a core shaft; a coil portion that is coupled to a distal end portion of the core shaft and is helically wound around a periphery of the core shaft; and a sound source that is provided in an inner side of the coil portion, in which the coil portion includes an open portion in which an interval of element wires constituting the coil portion itself is larger than that in another portion. With this configuration, the sound source is disposed in the inner side of the coil portion, and ultrasound that is generated from the sound source is emitted through the open portion to the periphery of the guide wire, so that it is possible to improve the mechanical strength of the guide wire as compared with that of a guide wire provided with a conventional sound source, and to emit ultrasound to the periphery of the guide wire more reliably.

(Configuration 2)

The guide wire according to Configuration 1, in which the sound source is provided in an inner side of the open portion. With this configuration, the propagation of the ultrasound emitted from the sound source is difficult to prevent by the element wires in the coil portion, so that it is possible to emit the ultrasound to the periphery of the guide wire more reliably.

(Configuration 3)

The guide wire according to Configuration 1 or 2, in which the coil portion has a bent shape that is bent relative to a longitudinal direction of the guide wire, and the open portion is formed in a range toward a rear from the bent portion in the coil portion.

(Configuration 4)

The guide wire according to Configuration 1 or 2 includes wiring that extends along an extending direction of the core shaft, in which a distal end of the wiring is positioned at the inner side of the coil portion, and the wiring supplies energy to the sound source.

(Configuration 5)

The guide wire according to Configuration 4, in which the coil portion has a bent shape that is bent relative to the longitudinal direction of the guide wire, and the distal end of the wiring is present rearward of the bent portion in the coil portion. The distal end of the wiring is positioned rearward of the bent portion, so that a mechanical load to be applied to the wiring due to warpage of the bent portion during the manipulation is suppressed.

(Configuration 6)

The guide wire according to Configuration 4 or 5, in which the wiring is an optical fiber, the guide wire further includes a light absorber that is provided in the inner side of the coil portion, in which the sound source is formed of the light absorber. With this configuration, the light absorber absorbs a pulse laser released from a distal end of the optical fiber and functions as a sound source, so that a device such as a vibration element does not need to be incorporated in the coil portion, and it is possible to reduce the diameter of the distal end of the guide wire.

(Configuration 7)

The guide wire according to Configuration 4 or 5, in which the wiring is a conductive wire, the sound source is a vibration element, and the vibration element is connected to a distal end of the conductive wire.

(Configuration 8) A guide wire includes: a core shaft; a coil portion that is coupled to a distal end portion of the core shaft and is helically wound around a periphery of the core shaft; an optical fiber that extends along an extending direction of the core shaft, and includes a distal end portion that is positioned at an inner side of the coil portion; and a light absorber that covers a surface of the coil portion, in which the coil portion includes an open portion in which an interval of element wires constituting the coil portion itself is larger than that in another portion, and a distal end of the optical fiber is positioned at an inner side of the open portion. With this configuration, the pulse laser released from the distal end of the optical fiber is absorbed by the light absorber through the open portion to generate ultrasound, which is emitted to the periphery of the guide wire. Therefore, as compared with a guide wire provided with a conventional sound source, it is possible to improve the mechanical strength, and to emit the ultrasound to the periphery of the guide wire in an excellent manner. Moreover, the distal end of the optical fiber is positioned at the inner side of the open portion, so that the released pulse laser is difficult to interfere with by the element wires in the coil portion, and it is possible to emit the ultrasound more reliably.

(Configuration 9)

The guide wire according to Configuration 8, in which the coil portion has a bent shape that is bent relative to a longitudinal direction of the guide wire, and the distal end of the optical fiber is present rearward of the bent portion in the coil portion. The distal end of the optical fiber is positioned rearward of the bent portion, so that a mechanical load to be applied to the optical fiber due to warpage of the bent portion during the manipulation is suppressed.

(Configuration 10)

The guide wire according to Configuration 8, in which the coil portion has a bent shape that is bent relative to the longitudinal direction of the guide wire, and the open portion is formed in a range toward a rear from the bent portion in the coil portion.

(Configuration 11)

The guide wire according to any one of Configurations 1 to 10, in which a gap between the element wires in the open portion is 0.13 mm or more and 0.33 mm or less. With this configuration, the gap is set to 0.13 mm or more, so that it is possible to maintain the ultrasonic sound pressure to be released through the open portion to the periphery of the guide wire comparatively high, and to emit the ultrasound more reliably. Moreover, the gap is set to 0.33 mm or less, so that a mechanical load to be applied to the open portion during the manipulation is suppressed.

(Configuration 12)

The guide wire according to any one of Configurations 1 to 11 includes a resin filled in the inner side of the coil portion. With the filled resin, it is possible to fix the position of the sound source provided in the inner side of the coil portion or the distal end of the optical fiber, and to prevent lowering of the emission of the ultrasound to the periphery of the guide wire due to a position shift thereof.

1. A guide wire comprising:
a core shaft;
a coil portion that is coupled to a distal end portion of the core shaft and that is helically wound around a periphery of the core shaft; and
a sound source that is provided in an inner side of the coil portion, wherein
the coil portion includes an open portion in which an interval of element wires constituting the coil portion itself is larger than that in another portion.

2. The guide wire according to claim 1, wherein the sound source is provided in an inner side of the open portion.

3. The guide wire according to claim 1, wherein
the coil portion has a bent shape that is bent relative to a longitudinal direction of the guide wire, and
the open portion is formed in a range toward a rear from the bent portion in the coil portion.

4. The guide wire according to claim 1, comprising wiring that extends along an extending direction of the core shaft, wherein
a distal end of the wiring is positioned at the inner side of the coil portion, and
the wiring supplies energy to the sound source.

5. The guide wire according to claim 4, wherein
the coil portion has a bent shape that is bent relative to the longitudinal direction of the guide wire, and
the distal end of the wiring is present rearward of the bent portion in the coil portion.

6. The guide wire according to claim 4, wherein the wiring is an optical fiber,
the guide wire further comprising a light absorber that is provided in the inner side of the coil portion, wherein the sound source is formed of the light absorber.

7. The guide wire according to claim 4, wherein
the wiring is a conductive wire,
the sound source is a vibration element, and
the vibration element is connected to a distal end of the conductive wire.

8. A guide wire comprising:
a core shaft;
a coil portion that is coupled to a distal end portion of the core shaft and that is helically wound around a periphery of the core shaft; and
an optical fiber that extends along an extending direction of the core shaft, and includes a distal end portion that is positioned at an inner side of the coil portion, and
a light absorber that covers a surface of the coil portion, wherein
the coil portion includes an open portion in which an interval of element wires constituting the coil portion itself is larger than that in another portion, and
a distal end of the optical fiber is positioned at an inner side of the open portion.

9. The guide wire according to claim 8, wherein
the coil portion has a bent shape that is bent relative to a longitudinal direction of the guide wire, and
the distal end of the optical fiber is present rearward of the bent portion in the coil portion.

10. The guide wire according to claim 8, wherein
the coil portion has a bent shape that is bent relative to the longitudinal direction of the guide wire, and
the open portion is formed in a range toward a rear from the bent portion in the coil portion.

11. The guide wire according to claim 1, wherein a gap between the element wires in the open portion is 0.13 mm or more and 0.33 mm or less.

12. The guide wire according to claim 1, comprising a resin filled in the inner side of the coil portion.

13. The guide wire according to claim 8, wherein the gap between the element wires in the open portion is 0.13 mm or more and 0.33 mm or less.

14. The guide wire according to claim 8, comprising a resin filled in the inner side of the coil portion.
